(19) United States
(12) Patent Application Publication
(10) Pub. No.: US 2025/0319103 A1
(43) Pub. Date: Oct. 16, 2025

(54) COMPOSITIONS FOR TREATMENT OF ANAL GLAND DISEASE

(71) Applicant: VETNIQUE LABS, LLC, Naperville, IL (US)

(72) Inventor: Jamy A. Bascharon, Naperville, IL (US)

(21) Appl. No.: 18/868,784

(22) PCT Filed: May 24, 2023
(86) PCT No.: PCT/US23/23423

§ 371 Date: Nov. 25, 2024

Related U.S. Application Data
(60) Provisional application No. 63/345,407, filed on May 24, 2022.

(51) Int. Cl. A61K 31/573; A61K 31/135; A61K 31/167; A61K 31/245; A61K 31/355; A61K 31/5375; A61K 36/28; A61K 36/899

(52) U.S. Cl. CPC A61K 31/573; A61K 31/135; A61K 31/167; A61K 31/245; A61K 31/355; A61K 31/5375; A61K 36/28; A61K 36/899

(57) ABSTRACT

This disclosure relates to a composition comprising an anti-inflammatory agent and an analgesic agent for treating anal gland disease. In particular, the disclosure relates to a composition formulated as a spray or wipe.

### RELATED APPLICATION

This application claims priority to U.S. Provisional Application No. 63/345,407, filed May 24, 2022, the contents of which are hereby incorporated by reference in their entirety.

### FIELD OF THE DISCLOSURE

This disclosure relates to a composition comprising an anti-inflammatory agent and an analgesic agent for treating anal gland disease. In particular, the disclosure relates to a composition formulated as a spray, cream, lotion, gel, fluid, or soap, or formulated into or integrated into a wipe.

### BACKGROUND OF THE DISCLOSURE

The anal glands (or anal sacs) are small glands found near the anus in many mammals, including pets such as dogs and cats. The two glands are located on either side of the anus, between the external and internal sphincter muscles. These glands secrete an oily discharge when the mammal defecates and this secretion is used for scent marking purposes. As the mammal defecates, the movement of the stool causes pressure over the glands, which secrete a few drops of liquid.

Many factors can cause the anal glands to not empty properly or to become inflamed and irritated, which can lead to various problems for the mammal. Some factors include poor quality stool (too soft or too small to exert pressure on the glands), allergies that may cause inflammation to the glands or the ducts where the liquid is secreted from, infection, and poor anatomy. When the glands do not empty regularly, the pressure builds up within the glands causing discomfort. If this continues an infection may set in and, if left unchecked, an abscess may develop which usually requires surgical treatment. Dogs or cats with severe ongoing anal gland problems may require complete surgical removal of the anal glands by a veterinarian.

The symptoms that a dog or cat with anal gland disease may display are: scooting the rear end on the ground, licking or biting at the rear, straining to defecate, giving away a very foul odor. defecating outside the litter box, etc. In severe cases, the sacs may abscess and rupture. Treatment usually involves having the glands manually emptied at a veterinarian's office or groomer. The standard recommendation for long-term treatment or prevention for animals that have repeated problems with their glands is a high fiber diet, which can help to add bulk and firm the stools.

### SUMMARY OF THE DISCLOSURE

Provided herein is a composition comprising a cortisone in an amount between about 0.1% to about 10% by weight of the total composition and an analgesic agent in an amount between about 1% to about 15% by weight of the total composition.

Provided herein is a composition comprising hydrocortisone in an amount between about 0.1% to about 10% by weight of the total composition and lidocaine in an amount between about 0.01% to about 15% by weight of the total composition. In some embodiments. a composition comprising hydrocortisone in an amount between about 0.5% to about 10% by weight of the total composition and lidocaine in an amount between about 0.1% to about 3% by weight of the total composition.

Provided herein is a composition comprising hydrocortisone in an amount between about 0.1% to about 10% by weight of the total composition and benzocaine in an amount between about 0.01% to about 15% by weight of the total composition. In some embodiments, a composition comprising hydrocortisone in an amount between about 0.5% to about 10% by weight of the total composition and benzocaine in an amount between about 0.1% to about 3% by weight of the total composition.

Provided herein is a composition comprising hydrocortisone in an amount between about 0.1% to about 10% by weight of the total composition and pramoxine in an amount between about 0.01% to about 15% by weight of the total composition. In some embodiments, a composition comprising hydrocortisone in an amount between about 0.5% to about 10% by weight of the total composition and pramoxine in an amount between about 0.1% to about 3% by weight of the total composition.

In some embodiments, one or more compositions disclosed may additionally comprise an antihistamine. In some embodiments, the antihistamine is diphenhydramine.

In some embodiments, one or more compositions disclosed herein may additionally comprise one or more soothing agents, odor reducing agents, and/or essential oils.

In some embodiments, the composition is formulated as a wipe. In other embodiments, the composition is formulated as a spray. In still other embodiments, the composition is formulated as a cream. lotion, gel, or fluid.

Further provided herein is a method of treatment or prevention of anal gland disease, the method comprising administering to a subject in need of such treatment an effective amount of one or more compositions disclosed herein.

### DETAILED DESCRIPTION OF THE DISCLOSURE

Provided herein is a composition for use in the treatment of anal gland disease, the composition comprising an anti-inflammatory agent to reduce anal itching and swelling and an analgesic agent to reduce anal pain. In particular embodiments, the composition comprises hydrocortisone as the anti-inflammatory agent and lidocaine as the analgesic agent. In other embodiments, the composition comprises hydrocortisone as the anti-inflammatory agent and benzocaine as the analgesic agent. In still other embodiments, the composition comprises hydrocortisone as the anti-inflammatory agent and pramoxine as the analgesic agent.

In the present disclosure, ranges and amounts can be expressed as “about” a particular value or range. The term “about” also includes the exact amount. For example, “about 5%” means “about 5%” and also “5%.” The term “about” can also refer to ±10% of a given value or range of values. Therefore, “about 5%” also means 4.5%- 5.5%, for example.

Hydrocortisone (11β,17α,21-trihydroxypregn-4-ene-3,20-dione; CAS RN: 50-23-7) is a corticosteroid commonly supplied in medication. In some embodiments, the disclosure provides a composition, wherein hydrocortisone is in the amount of about 0.1% to about 10% weight of the composition (wt %). In other embodiments, hydrocortisone is in the amount of about 0.5 to about 5 wt %. In other embodiments, hydrocortisone is in the amount of about 0.5 to about 1 wt %. In still other embodiments. hydrocortisone is in the amount of about 1 wt %.

In certain embodiments, the analgesic agent is lidocaine, pramoxine, or benzocaine.

Lidocaine (2-(diethylamino)-N-(2,6-dimethylphenyl) acetamide; CAS RN: 137-58-6) is a local anesthetic that works by causing temporary numbness/loss of feeling in the skin and mucous membranes. In some embodiments, the disclosure provides a composition, wherein lidocaine is in the amount of about 0.01 to about 15 wt %. In other embodiments, lidocaine is in the amount of about 0.1 to about 10 wt %. In another embodiment, lidocaine is in the amount of about 0.1 to about 3 wt %. In other embodiments, the lidocaine is in the amount of about 3 wt %. The lidocaine can be provided as a hydrochloride (HCl) salt. Thus, in some embodiments, the lidocaine of the disclosure is lidocaine hydrochloride (lidocaine HCl).

In some embodiments, the disclosure provides a composition, wherein the hydrocortisone is in the amount of about 0.1 to about 10 wt % and lidocaine is in the amount of about 0.01% to about 15% by weight of the total composition. In some embodiments. the disclosure provides a composition, wherein hydrocortisone is in the amount of about 0.1 wt % and lidocaine in is in the amount of about 3 wt %.

Benzocaine (ethyl 4-aminobenzoate; CAS RN: 94-09-7) is also a local anesthetic. In some embodiments, the disclosure provides a composition, wherein benzocaine is in the amount of about 0.01 to about 15 wt %. In other embodiments. benzocaine is in the amount of about 0.1 to about 10 wt %. In another embodiment, benzocaine is in the amount of about 0.1 to about 3 wt %. In other embodiments, the benzocaine is in the amount of about 3 wt %.

In some embodiments, the disclosure provides a composition, wherein the hydrocortisone is in the amount of about 0.1 to about 10 wt % and benzocaine is in the amount of about 0.01% to about 15% by weight of the total composition. In some embodiments, the disclosure provides a composition, wherein hydrocortisone is in the amount of about 1 wt % and benzocaine in is in the amount of about 0.1 to about 3 wt %.

Pramoxine (4-|3-(4-butoxyphenoxy)propyl|morpholine; CAS RN: 140-65-8) is also a local anesthetic. In some embodiments, the disclosure provides a composition, wherein pramoxine is in the amount of about 0.01 to about 15 wt %. In other embodiments, pramoxine is in the amount of about 0.1 to about 10 wt %. In another embodiment, pramoxine is in the amount of about 0.1 to about 3 wt %. In other embodiments, the pramoxine is in the amount of about 3 wt %. The pramoxine can be provided as a hydrochloride (HCl) salt. Thus, in some embodiments, the pramoxine of the disclosure is pramoxine hydrochloride (pramoxine HCl).

In some embodiments, the disclosure provides a composition, wherein the hydrocortisone is in the amount of about 0.5 to about 10 wt % and pramoxine is in the amount of about 0.01% to about 15% by weight of the total composition. In some embodiments, the disclosure provides a composition, wherein hydrocortisone is in the amount of about 1 wt % and pramoxine in is in the amount of about 0.1 to about 3 wt %.

In some embodiments, the composition further comprises an antihistamine, wherein the antihistamine is in the amount of about 0.01 to about 15 wt %. In other embodiments, the composition further comprises an antihistamine, wherein the antihistamine is in the amount of about 0.1 to about 3 wt %. In some embodiments, the antihistamine is diphenhydramine.

In some embodiments, the composition further comprises an odor reducing or trapping agent. Suitable odor reducing or trapping agents may include any composition that traps odor causing compounds, such as amines, sulfides, mercaptans. In one embodiment, the odor reducing or trapping agent is the water-based, semi-rigid, concave molecular structures sold as Ordenone® by Belle-Aire Fragrances, Inc., Mundelein, Ill.

In some embodiments, the disclosure provides a composition, wherein the odor reducing or trapping agent is in the amount of about 0.1 to about 5 wt %. In other embodiments, the odor reducing or trapping agent is in the amount of about 0.5 to about 5 wt %. In other embodiments, the odor reducing or trapping agent is in the amount of about 0.1 to about 1 wt %. In another embodiment, the odor reducing or trapping agent is in the amount of about 0.5 to about 1 wt %. In other embodiments, the odor reducing or trapping agent is in the amount of about 0.5 wt %.

In some embodiments, the composition further comprises one or more soothing agents. The term “soothing agent” means a molecule which helps in reducing the discomfort of the skin. Suitable soothing agents include tocopheryl acetate and/or other forms of vitamin E, chamomile extract, aloe, avocado oil, green tea extract. hops extract, colloidal oatmeal, calamine, cucumber extract, sodium palmate, sodium palm kernelate, Butyrospermum parkii (i.e., shea butter), Menthe piperita (i.e., peppermint) leaf oil, sericin, pyridoxine (a form of vitamin B6), retinyl palmitate and/or other forms of vitamin A, lauryl laurate, hyaluronic acid, aloe barbadensis leaf juice powder, Euterpe oleracea (i.e., acai berry) fruit extract, riboflavin (i.e., vitamin B2), thiamin HCl and/or other forms of vitamin B1, and any combinations thereof. In some embodiments, the composition comprises tocopheryl acetate and chamomile extract.

In some embodiments, the disclosure provides a composition, wherein the soothing agent is in the amount of about 0.001 to about 1 wt %. In other embodiments, the soothing agent is in the amount of about 0.01 to about 1 wt %. In other embodiments, the soothing agent is in the amount of about 0.1 to about 1 wt %. In another embodiment, the soothing agent is in the amount of about 0.01 wt %. In other embodiments, the soothing agent is in the amount of about 0.1 wt %. In other embodiments, tocopheryl acetate is in the amount of about 0.01 wt % and chamomile extract is in the amount of about 0.1 wt %.

In some embodiments, the composition further comprises an oat avenanthramide extract. The oat avenanthramide extract is a natural anti-oxidant and anti-irritant that reduces redness, inflammation and itching of the skin. In some embodiments, the disclosure provides a composition, wherein the oat avenanthramide extract is in the amount of about 0.1 to about 5 wt %. In other embodiments. the oat avenanthramide extract is in the amount of about 0.5 to about 5 wt %. In another embodiment, the oat avenanthramide extract is in the amount of about 0.5 to about 1 wt %. In other embodiments, the oat avenanthramide extract is in the amount of about 0.5 wt %.

In other embodiments, the composition may comprise additional ingredients as appropriate that would be well known to one of ordinary skill in the art such as penetration enhancers, humectants, lubricants, color, fragrance, preservatives, antioxidants, chelators, neutralizers, astringents, binders, catalysts, stabilizers, emollients, emulsifiers, surfactants, essential oils. plant/botanical extracts, conditioners, film formers, gelling agents, foaming agents, exfoliants, vitamins, minerals, pH adjusters, proteins, peptides, tactile enhancers, saccharides, solvents or any combination thereof.

In some embodiments, the composition further comprises a humectant such as propylene glycol. In some embodiments, the composition further comprises an emulsifier such as PEG-75 lanolin. In some embodiments, the composition further comprises a preservative such as kalama benzyl alcohol. In some embodiments, the composition further comprises an astringent such as alcohol. In some embodiments, the composition further comprises a fragrance.

In some embodiments, the composition disclosed herein may be formulated as a spray, wipe, cream, lotion, gel. fluid/liquid, soap, or any other form known in the art. In some embodiments, the composition is formulated as a spray. In some embodiments, the composition is formulated as a wipe. One of skill in the art recognizes that the wipe comprises an absorbent substrate and the composition of the disclosure, wherein the substrate is pre-moistened with the composition. In some embodiments, the wipe may take the form of a flexible substrate, and in certain embodiments may be made from a woven or a non-woven fabric substrate.

In some embodiments, the disclosure provides methods of treating or preventing anal gland disease, the method comprising administering to a subject in need of such treatment an effective amount of one or more of compositions of the disclosure. Any disclosed composition, or a combination of compositions may be used in these methods. Administration of a spray, a medicated wipe, or an aerosol may comprise delivering one or more compositions of the disclosure topically to a subject. In some embodiments, administering comprises application of a spray, medicated wipe, or aerosol comprising one or more compositions of the disclosure to the subject's anal glands. In the embodiment of administration of a wipe, the wipe is applied to the anal region or other parts of the pet body in sufficient manner so as to deposit one or more compositions of the disclosure on the surface to be treated, and is then discarded in the trash after use. In some embodiments, wipes or spray comprising the disclosed composition may be applied to a pet's paws, coat, face, ears, and anal area.

The disclosure provides methods as described above, wherein the subject is a pet. In one embodiment, the subject is a dog or a cat. In another embodiment, the subject is a dog. In other embodiment, the subject is a cat.

In some embodiments, in the methods as described above, the composition is administered over the course of a period of time. In some embodiments, the composition may be applied on a daily basis. either once a day or several times per day, over the course of days, weeks, months or years. In some embodiments, in the methods as described above, the administration is episodically, for example on an as needed basis when the subject is experiencing anal gland problems for a short-term period of several months.

The compositions described herein will generally be used in an amount effective to achieve the intended result, for example in an amount effective to treat or prevent the anal gland disease. By therapeutic benefit is meant eradication or amelioration of the anal gland disease and/or eradication or amelioration of one or more of the symptoms associated with the anal gland disease such that the subject shows an improvement in feeling or condition, notwithstanding that the subject may still be afflicted with the disease. Therapeutic benefit also generally includes halting or slowing the progression of the anal gland disease, regardless of whether improvement is realized.

### EXAMPLES

The compositions and methods of the disclosure are further illustrated by the following examples, which are not to be construed as limiting the disclosure in scope or spirit to the specific procedures and compositions described in them.

Example 1: A composition is formulated into a non-pressurized spray by mixing the ingredients at room temperature to obtain a solution, followed by including the solution into a spray bottle.

Representative compositions that are discharged per pump are listed in Table 1, Table 2, and in Table 3.

TABLE 1

| | |
|---|---|
| Amount (mg/pump) | wt % |

Spray
serving = 1 pump (0.305 g/pump) (390 pumps in 4 oz)
Actives

| | | |
|---|---|---|
| Hydrocortisone | 0.0033 | 1.07% |
| Lidocaine HCl | 0.0101 | 3.31% |

Inactives

| | | |
|---|---|---|
| Deionized Water | 0.0545 | 17.88% |
| Propylene Glycol | 0.2198 | 72.08% |
| Glycerin | 0.0092 | 3.00% |
| Tocopheryl Acetate | 0.0000 | 0.01% |
| Oat Avenanthramides 100 ppm | 0.0015 | 0.50% |
| Ordenone #209598 | 0.0015 | 0.50% |
| PEG-75 Lanolin | 0.0031 | 1.00% |
| Fragrance: White Tea & Ginger | 0.0009 | 0.30% |
| Chamomile Extract | 0.0003 | 0.10% |
| Kalama Benzyl Alcohol | 0.0008 | 0.25% |

TABLE 2

| | |
|---|---|
| Amount (mg/pump) | wt % |

Spray
serving = 1 pump (0.305 g/pump) (390 pumps in 4 oz)
Actives

| | | |
|---|---|---|
| Hydrocortisone | 0.0033 | 1.07% |
| Bezocaine | 0.0101 | 3.31% |

Inactives

| | | |
|---|---|---|
| Deionized Water | 0.0545 | 17.88% |
| Propylene Glycol | 0.2198 | 72.08% |
| Glycerin | 0.0092 | 3.00% |
| Tocopheryl Acetate | 0.0000 | 0.01% |
| Oat Avenanthramides 100 ppm | 0.0015 | 0.50% |
| Ordenone #209598 | 0.0015 | 0.50% |
| PEG-75 Lanolin | 0.0031 | 1.00% |
| Fragrance: White Tea & Ginger | 0.0009 | 0.30% |
| Chamomile Extract | 0.0003 | 0.10% |
| Kalama Benzyl Alcohol | 0.0008 | 0.25% |

TABLE 3

| | |
|---|---|
| Amount (mg/pump) | wt % |

Spray
serving = 1 pump (0.305 g/pump) (390 pumps in 4 oz)
Actives

| | | |
|---|---|---|
| Hydrocortisone | 0.0033 | 1.07% |
| Pramoxine HCl | 0.0101 | 3.31% |
| Diphenhydramine | 0.0033 | 1.00% |

Inactives

| | | |
|---|---|---|
| Deionized Water | 0.0545 | 17.88% |
| Propylene Glycol | 0.2198 | 72.08% |
| Glycerin | 0.0092 | 3.00% |
| Tocopheryl Acetate | 0.0000 | 0.01% |
| Oat Avenanthramides 100 ppm | 0.0015 | 0.50% |
| Ordenone #209598 | 0.0015 | 0.50% |
| PEG-75 Lanolin | 0.0031 | 1.00% |
| Fragrance: White Tea & Ginger | 0.0009 | 0.30% |
| Chamomile Extract | 0.0003 | 0.10% |
| Kalama Benzyl Alcohol | 0.0008 | 0.25% |

Example 2: A composition is formulated into a wipe by mixing the ingredients at room temperature to obtain a solution, followed by soaking an absorbent wipe substrate in the solution until the solution is absorbed onto the wipe. Representative compositions that are present per wipe are listed in Table 3, Table 4, and Table 5.

TABLE 3
Wipe

| | |
|---|---|
| Amount (mg/wipe) | wt % |

Actives

| | | |
|---|---|---|
| Hydrocortisone | 1,931 | 1.07% |
| Lidocaine HCl | 5,955 | 3.31% |

Inactives

| | | |
|---|---|---|
| Propylene Glycol | 118,440 | 65.80% |
| DI Water | 37,186 | 20.66% |
| SD Alcohol 40B | 12,600 | 7% |
| PEG-75 Lanolin | 900 | 0.50% |
| Oat Avenathramides 100 ppm | 900 | 0.50% |
| Ordenone #209598 | 900 | 0.50% |
| White Tea & Ginger | 540 | 0.30% |
| Kalama Benzyl Alcohol | 450 | 0.25% |
| Chamomile Extract | 180 | 0.10% |
| Tocopheryl Acetate | 18 | 0.01% |

TABLE 4
Wipe

| | |
|---|---|
| Amount (mg/wipe) | wt % |

Actives

| | | |
|---|---|---|
| Hydrocortisone | 1,931 | 1.07% |
| Benzocaine | 5,955 | 3.31% |

Inactives

| | | |
|---|---|---|
| Propylene Glycol | 118,440 | 65.80% |
| DI Water | 37,186 | 20.66% |
| SD Alcohol 40B | 12,600 | 7% |
| PEG-75 Lanolin | 900 | 0.50% |
| Oat Avenathramides 100 ppm | 900 | 0.50% |
| Ordenone #209598 | 900 | 0.50% |
| White Tea & Ginger | 540 | 0.30% |
| Kalama Benzyl Alcohol | 450 | 0.25% |
| Chamomile Extract | 180 | 0.10% |
| Tocopheryl Acetate | 18 | 0.01% |

TABLE 5
Wipe

| | Amount (mg/wipe) | wt % |
|---|---|---|
| Actives | | |
| Hydrocortisone | 1,931 | 1.07% |
| Pramoxine HCl | 5,955 | 3.31% |
| Diphenhydramine | 0.0033 | 1.00% |

Inactives

| | | |
|---|---|---|
| Propylene Glycol | 118,440 | 65.80% |
| DI Water | 37,186 | 20.66% |
| SD Alcohol 40B | 12,600 | 7% |
| PEG-75 Lanolin | 900 | 0.50% |
| Oat Avenathramides 100 ppm | 900 | 0.50% |
| Ordenone #209598 | 900 | 0.50% |
| White Tea & Ginger | 540 | 0.30% |
| Kalama Benzyl Alcohol | 450 | 0.25% |
| Chamomile Extract | 180 | 0.10% |
| Tocopheryl Acetate | 18 | 0.01% |

It is understood that the examples and embodiments described herein are for illustrative purposes only. Unless clearly excluded by the context, all embodiments disclosed for one aspect of the invention can be combined with embodiments disclosed for other aspects of the invention, in any suitable combination. It will be apparent to those skilled in the art that various modifications and variations can be made to the present invention without departing from the scope of the invention. Thus. it is intended that the present invention cover the modifications and variations of this invention provided they come within the scope of the appended claims and their equivalents.

1. A composition comprising hydrocortisone in an amount between about 0.1% to about 10% by weight of the total composition and pramoxine in an amount between about 0.1% to about 15% by weight of the total composition.

2. The composition of claim 1, wherein hydrocortisone is in the amount of about 1% by weight of the total composition and pramoxine in is the amount of about 3% by weight of the total composition.

3. The composition of claim 1, wherein the pramoxine is pramoxine hydrochloride.

4. The composition of claim 1, further comprising diphenhydramine in an amount of about 1% by weight of the total composition.

5. A composition comprising hydrocortisone in an amount between about 0.1% to about 10% by weight of the total composition and benzocaine in an amount between about 1% to about 15% by weight of the total composition.

6. The composition of claim 1 further comprising an odor reducing agent.

7. The composition of claim 6, wherein the odor reducing agent is in an amount between about 0.1% to about 5% by weight of the total composition.

8. The composition of claim 7, wherein the odor reducing agent is in the amount of about 0.5% by weight of the total composition.

9. The composition of claim 1 further comprising one or more soothing agents.

10. The composition of claim 9, wherein the one or more soothing agents is selected from tocopheryl acetate and chamomile extract.

11. The composition of claim 10, wherein tocopheryl acetate is in the amount of about 0.01% by weight of the total composition and chamomile extract is in the amount of about 0.1% by weight of the total composition.

12. The composition of any one of claims 1-14 further comprising an oat avenanthramide extract.

13. The composition of claim 12, wherein the oat avenanthramide extract is in the amount of about 0.1% to about 5% by weight of the total composition.

14. The composition of claim 1 further comprising one or more essential oils.

15. The composition of claim 1, wherein the composition is formulated as a spray.

16. The composition of claim 1, wherein the composition is formulated as a wipe.

17. The composition of claim 16, wherein the wipe comprises an absorbent wipe substrate.

18. A composition comprising hydrocortisone in an amount between about 0.5% to about 10% by weight of the total composition and lidocaine in an amount between about 1% to about 15% by weight of the total composition.

19. A method of treatment or prevention of anal gland disease, the method comprising administering to a subject in need of such treatment an effective amount of one or more compositions of claim 1.

20. The method of claim 19, wherein the subject is a dog or a cat.
